(19) United States
(12) Patent Application Publication
(10) Pub. No.: US 2026/0174370 A1
(43) Pub. Date: Jun. 25, 2026

(54) BIOMETRIC ACQUISITION DEVICE

(71) Applicant: Nippon Telegraph and Telephone Corporation, Tokyo (JP)

(72) Inventors: Kento Watanabe, Tokyo (JP); Kenichi Matsunaga, Tokyo (JP)

(21) Appl. No.: 18/856,832

(22) PCT Filed: Jun. 6, 2022
(86) PCT No.: PCT/JP2022/022727

§ 371 Date: Oct. 14, 2024

(51) Int. Cl. A61B 5/257; A61B 5/00; A61B 5/318

(52) U.S. Cl. CPC A61B 5/257; A61B 5/0028; A61B 5/6843; A61B 5/318

(57) ABSTRACT

A biological signal acquisition device includes: an electrode and an electrode configured to be in contact with a human body and configured to acquire a biological signal emitted by the human body when the electrodes are in contact with the human body; and an elastic body configured to press the electrode against the human body when the electrode is in contact with the human body. The biological signal acquisition device further includes an elastic body that presses the electrode against the human body when the electrode is in contact with the living body. The biological signal acquisition device further includes a circuit board 16 that acquires a biological signal using the electrode and the electrode.

### CROSS-REFERENCE TO RELATED APPLICATIONS

This application is a national phase entry of PCT Application No. PCT/JP2022/022727, filed on Jun. 6, 2022, which application is hereby incorporated herein by reference.

### TECHNICAL FIELD

The present invention relates to a biological signal acquisition device that acquires a biological signal.

### BACKGROUND

A biological signal emitted from a living body such as a person (for example, a signal constituting at least a part of a waveform of an electrocardiogram or an electromyogram, or a signal of an electroencephalogram) is measured by two electrodes in contact with the living body. Here, the signal obtained by the plurality of electrodes includes a noise component in addition to a signal component, which is a biological signal. Thus, various circuits that amplify the signal and also remove the noise component are provided at stages subsequent to the plurality of electrodes (Non Patent Literature 1).

### CITATION LIST

### Non Patent Literature

Non Patent Literature 1: Ting-Wei Wang, Shien-Fong Lin, “Negative Impedance Capacitive Electrode for ECG Sensing Through Fabric Layer,” in IEEE Transactions on Instrumentation and Measurement, vol. 70, pp. 1-8, 2021, Art no. 4002308, doi: 10.1109/TIM.2020.3045187.

### SUMMARY

### Technical Problem

Since the SN ratio, which is the ratio between the signal component and the noise component, is affected by the impedance between the living body and the electrode, it is desired to improve the contact state between the living body and the electrodes. However, the contact state between the living body and the electrodes is not always favorable due to the shape of the outer surface of the living body and/or the change in the shape caused by the motion of the living body.

An object of embodiments of the present invention is to improve the contact state between a living body and an electrode.

### Solution to Problem

In order to solve the above problems, a biological signal acquisition device according to embodiments of the present invention includes: a first electrode and a second electrode configured to be in contact with a living body and configured to acquire a biological signal emitted by the living body when the first electrode and the second electrode are in contact with the living body; and a first elastic body configured to press the first electrode against the living body when the first electrode is in contact with the living body.

### Advantageous Effects

According to embodiments of the present invention, a contact state between a living body and an electrode is improved.

### BRIEF DESCRIPTION OF THE DRAWINGS

FIG. 1 is a plan view of a biological signal acquisition device according to a first embodiment of the present invention as viewed from a human body side.

FIG. 2 is a cross-sectional view along line A-A of FIG. 1.

FIG. 3 is a cross-sectional view when the biological signal acquisition device is attached to a human body.

FIG. 4 is a plan view of a biological signal acquisition device according to a second embodiment of the present invention as viewed from a human body side.

FIG. 5 is a cross-sectional view along line B-B of FIG. 4.

FIG. 6 is a cross-sectional view of a biological signal acquisition device according to a third embodiment of the present invention.

FIG. 7 is a circuit diagram of a circuit mounted on a circuit board of the third embodiment of the present invention.

FIG. 8 is a circuit diagram of a circuit mounted on the circuit board of the third embodiment of the present invention.

FIG. 9 is a cross-sectional view of a biological signal acquisition device according to a modification.

### DETAILED DESCRIPTION OF ILLUSTRATIVE EMBODIMENTS

Hereinafter, embodiments of the present invention will be described below with reference to the drawings.

### First Embodiment

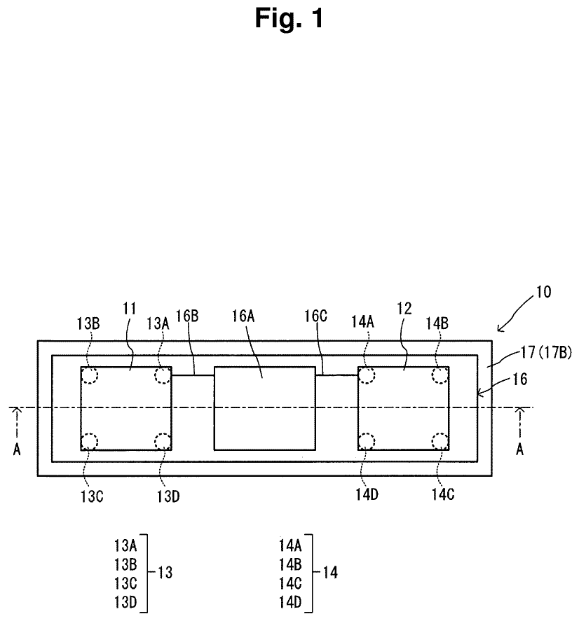

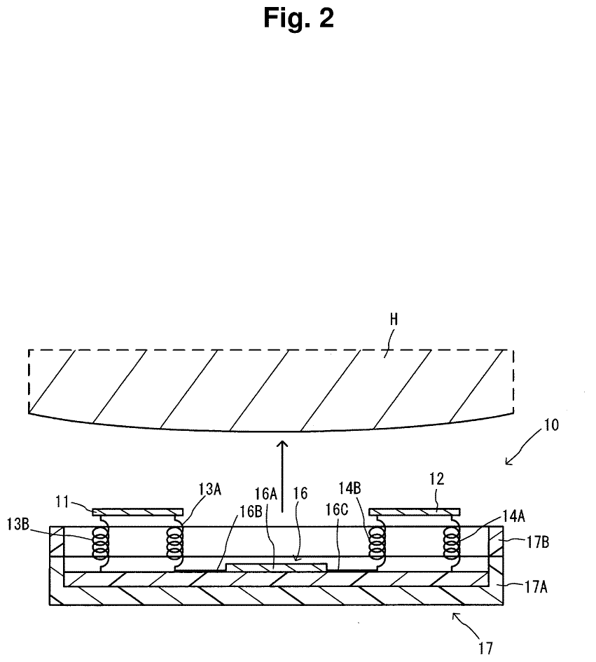

A biological signal acquisition device 10 according to a first embodiment illustrated in FIGS. 1 and 2 is attached to a predetermined portion of a human body H as a living body, and is configured to acquire a biological signal emitted by the human body H. Here, the biological signal is a biological signal used for the electrocardiogram.

The biological signal acquisition device 10 includes electrodes 11 and 12 that acquire biological signals, and elastic bodies 13 and 14 that press the electrodes 11 and 12 against the human body H, respectively. The biological signal acquisition device 10 further includes a circuit board 16 that acquires a biological signal by the electrodes 11 and 12 and outputs the biological signal, and a housing 17 that houses the circuit board 16 and is attached to the human body H.

The electrodes 11 and 12 come into contact with the human body H to acquire a biological signal from the human body H. The contact between the electrodes 11 and 12 and the human body H may be in direct contact or indirect contact via clothing or the like.

The electrodes 11 and 12 are formed in a rectangular sheet shape. The electrodes 11 and 12 have flexibility. The electrodes 11 and 12 are made from, for example, a metal thin film. The electrodes 11 and 12 made from a metal thin film may be reinforced by a resin material having elasticity. The electrodes 11 and 12 may be made of a conductive plastic or a conductive fiber material. The electrodes 11 and 12 are supported by the elastic bodies 13 and 14 provided on the circuit board 16, respectively at positions closer to the human body H than the circuit board 16 with a space from the circuit board 16.

The elastic body 13 supporting the electrode 11 includes a set of a plurality of (here, four) springs 13A to 13D. The elastic body 13 as a whole includes one end (one end of each of the springs 13A to 13D) fixed to the electrode 11 and the other end (one end of each of the springs 13A to 13D) fixed to the circuit board 16.

The elastic body 14 has a configuration similar to that of the elastic body 13 except for supporting the electrode 12. That is, the elastic body 14 includes a set of a plurality of springs 14A to 14D, and includes, as a whole, one end fixed to the electrode 12 and the other end fixed to the circuit board 16.

Various circuits including a processing circuit 16A such as a microcomputer are mounted on the circuit board 16. Here, the spring 13A includes a conductor electrically connected to the electrode 12 and the circuit board 16, and an insulating film partially covering the conductor. That is, the spring 13A also functions as a wiring that electrically connects the electrode 12 and the circuit board 16 (more specifically, a wiring pattern 16B connected to the processing circuit 16A). Similarly, the spring 14A also functions as a wiring that electrically connects the electrode 12 and the circuit board 16 (more specifically, a wiring pattern 16C connected to the processing circuit 16A). The potential of the electrode 11 and the potential of the electrode 12 are input to the processing circuit 16A via the springs 13A and 14A. Here, each potential of the electrodes 11 and 12 is a potential when any portion of the human body H is connected to a reference potential. The input of each potential to the processing circuit 16A may also be referred to as an input of a potential difference between the electrode 11 and the electrode 12. The signal represented by the potential difference, that is, the signal acquired by the electrodes 11 and 12 may include a signal component including a biological signal and a noise component.

The processing circuit 16A includes an amplifier that amplifies a signal represented by a potential difference between the electrode 11 and the electrode 12, and also includes appropriately a filter circuit that removes a noise component from the signal to extract, from the signal, a biological signal that is a signal component. With such a configuration, the processing circuit 16A acquires the biological signal using the electrodes 11 and 12. The processing circuit 16A externally outputs the acquired biological signal to a device that displays an electrocardiogram or the like.

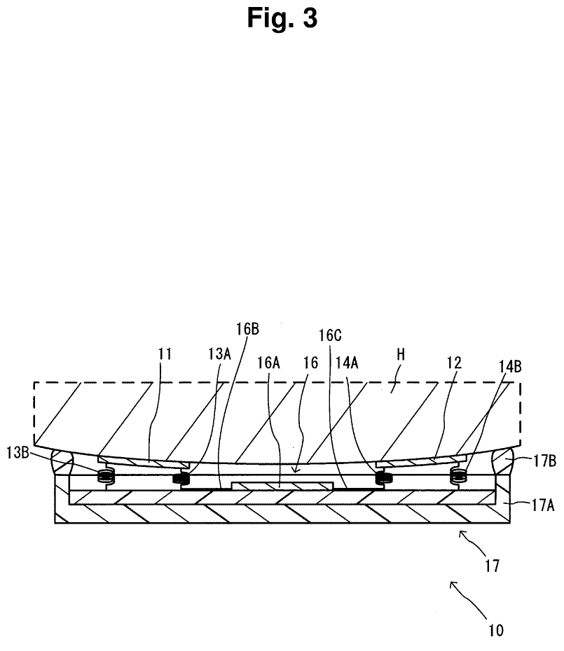

The housing 17 includes a box-shaped main body 17A having an opening on the human body H side, and a cylindrical body 17B fixed to the opening of the main body 17A and made of a flexible tubular sponge or the like. The circuit board 16 is fixed to the housing 17, that is, the bottom of the main body 17A. The housing 17 is attached to a predetermined portion of the human body H by a belt (not illustrated) or the like. At this time, the deformation of the cylindrical body 17B (see also FIG. 3) suppresses biting of the housing 17 into the human body H.

As illustrated in FIG. 3, when the housing 17 is attached to the human body H, that is, when the biological signal acquisition device 10 is attached to the human body H, the electrodes 11 and 12 come into contact with the human body H. This contact presses the electrodes 11 and 12 toward the circuit board 16 so that the elastic bodies 13 and 14 expand and contract. The expanded and contracted elastic bodies 13 and 14 press the electrodes 11 and 12 against the human body H by their elastic force. This pressing can improve the contact state between the human body H and the electrodes 11 and 12. In particular, in the present embodiment, the electrodes 11 and 12 have flexibility, so that the electrodes 11 and 12 can be deformed following the shape of the body surface of the human body H to improve the contact state.

The improvement of the contact state includes an increase in the contact area of the electrodes 11 and 12 and suppression of a decrease in the contact area when the shape of the outer surface of the human body H changes due to the motion (including respiration) of the human body H or the like. As a result, the impedance between the body surface and the electrode can be kept low, and as a result, the SN ratio of the signal input to the processing circuit 16A is improved (details will be described below). Furthermore, the improvement of the contact state includes reducing a difference between the contact area between the human body H and the electrode 11 and the contact area between the human body H and the electrode 12. As a result, the difference between the impedance between the body surface and the electrode 11 and the impedance between the body surface and the electrode 12 can be reduced, and as a result, decrease in a common-mode rejection ratio (CMRR) to be described below, that is, deterioration in the SN ratio of the signal amplified by the processing circuit 16A is suppressed.

The outer surface of the human body H is generally a curved surface. Therefore, depending on the structure of the elastic body 13, the outer peripheral portion (side or corner) of the electrode 11 is easily separated from the human body H. In the present embodiment, as illustrated in FIGS. 1 and 2, the springs 13A to 13D included in the elastic body 13 are connected to corresponding ones of all corners of the quadrangular electrode 11, so that separation of the outer peripheral portion of the electrode 11 from the human body H is suppressed, whereby the contact area is secured, and the contact state is improved. The same applies to the electrode 12 and the elastic body 14.

The electrodes 11 and 12 may have rigidity so as not to be deformed at the time of contact with the human body H. Even in such a case, by the electrodes 11 and 12 being pressed against the human body H, for example, the human body H can be deformed in accordance with the shapes of the electrodes 11 and 12, improving the contact state.

Securing the contact area to lower the impedance between the body surface and the electrode contributes to suppression of deterioration of the SN ratio of the signal represented by the potential difference between the electrode 11 and the electrode 12. Specifically, when the impedance between the body surface and the electrode is Ze, the input impedance of the processing circuit 16A of the circuit board 16 is Zi, and the biological potential of the human body H is Vh, the potential Vin received by the processing circuit 16A is derived by Formula (1) stated below. That is, the potential Vin is divided according to the impedance between the body surface and the electrode. When the contact area between the electrode 11 or 12 and the human body H decreases to increase the impedance between the body surface and the electrode, the potential Vin attenuates due to this division of pressure. In such a case, the noise component of the signal represented by the potential difference between the electrode 11 and the electrode 12 becomes relatively large, and as a result, the SN ratio deteriorates. On the other hand, by securing the contact area between the electrode 11 or 12 and the human body H as in the present embodiment, attenuation of Vin is suppressed to improve the SN ratio.

$$Vin = \{ Zi / ( Ze + Zi ) \} * Vh \qquad ( 1 )$$

The processing circuit 16A of the circuit board 16 may be a circuit that differentially amplifies the potential difference between the electrode 11 and the electrode 12. In such a case, the common mode noise generated in common at the electrode 11 and the electrode 12 is removed by differential amplification. This removal performance is indicated by CMRR. The CMRR is maximized when the impedance between the body surface and the amplifier from the body surface of the human body H to the amplifier input is the same at the electrode 11 and the electrode 12. However, when a pair of conventional electrodes, which do not include an elastic body, is used, a situation where the impedances between the body surface and the electrodes are different at the electrode 11 and the electrode 12 may occur due to the difference of the shape of the contact portion between the human body H and the shape with the electrode 11 and the contact portion between the human body H with the electrode 12, or change of the shape of the contact portion caused by the motion of the human body H or the like. The difference in impedance between the body surface and the electrode changes the in-phase noise components, which deteriorates CMRR. As a method of coping with such a problem, a method of adaptively adding an additional resistance to the subsequent stage of the electrode at which the impedance between the body surface and the electrode is lower, that is, the electrode in a better contact state to make the impedances between the body surface the electrode at the electrodes closer to each other can be considered. In addition, there is also a method in which a negative impedance circuit is provided immediately subsequent to each electrode, and the parameters thereof are controlled, so that the impedances between the body surface and the electrode at the electrodes are made close to each other. However, the addition of the resistor in the former method increases noise including thermal noise, degrading the signal. In addition, the negative impedance circuit increases the power consumption, and the range of the impedance that can be controlled is limited. In this embodiment, the contact area between the electrode 11 and the human body H and the contact area between the electrode 12 and the human body H can be brought close to each other by the elastic bodies 13 and 14, so that a difference in impedance between the body surface and the electrodes can be reduced, and a good CMRR can be obtained without using the resistance or the impedance circuit, that is, without generating extra noise or power consumption.

In the above-described embodiment, the electrode 11 is electrically connected to the circuit board 16 via the spring 13A, which is a part of the elastic body 13. As a result, the potential of the electrode 11 is input via the elastic body 13. However, the electrode 11 and the processing circuit 16A may be electrically connected by a wiring provided separately from the elastic body 13. However, with the former configuration, the number of parts is reduced, and the efficiency of the manufacturing process is improved. The same applies to the electrode 12 and the elastic body 14 (the spring 14A).

### Second Embodiment

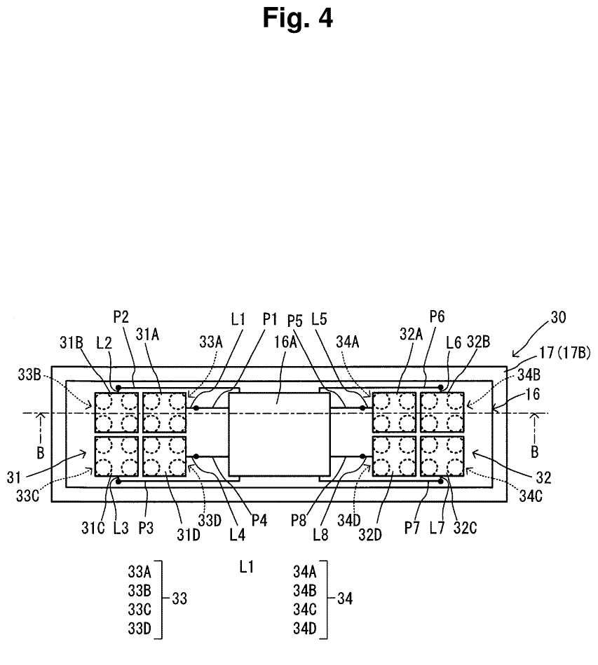

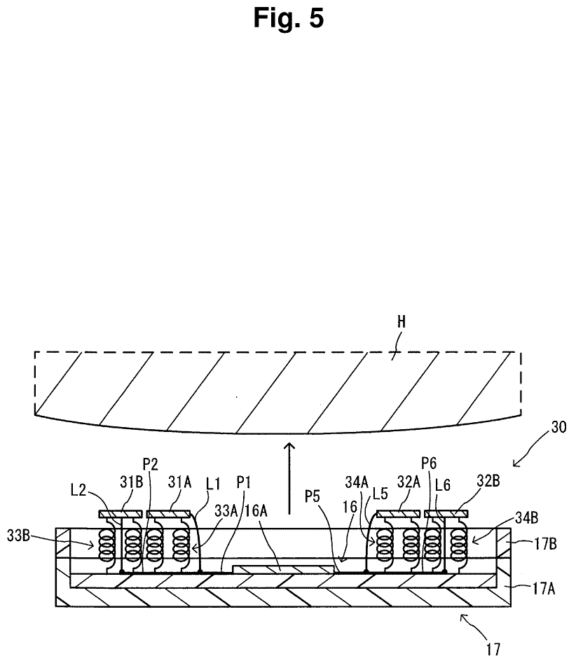

As illustrated in FIGS. 4 and 5, a biological signal acquisition device 30 according to a second embodiment includes electrodes 31 and 32 and elastic bodies 33 and 34 instead of the electrodes 11 and 12 and the elastic bodies 13 and 14 in the first embodiment. Hereinafter, differences from the first embodiment will be described. For the description not mentioned below, the description of the first embodiment is referred to as appropriate.

The electrode 31 includes four electrodes 31A to 31D. The electrodes 31A to 31D are regularly arranged, that is, arranged in an array. Here, the electrodes 31A to 31D are arranged in a two-dimensional array. With the electrodes 31A to 31D, the electrode 31 can be brought into contact with the human body H more flexibly according to the shape of the surface of the human body H. The electrode 31 may include any number of electrodes, and the number may be 2 or more. The elastic body 33 includes a set of elastic bodies 33A to 33D that support the electrodes 31A to 31D, respectively. Each of the elastic bodies 33A to 33D includes four springs. The set of the electrode 31 and the elastic body 33 has a structure in which the set of the electrode 11 and the elastic body 13 (springs 13A to 13D) of the first embodiment is divided into small pieces, and four small sets are arranged in an array. For example, a set of the electrode 31A and the elastic body 33A is one of the four sets. Therefore, the detailed description of the electrodes 31A to 31D and the elastic bodies 33A to 33D is similar to that provided above.

Similarly to the electrode 31, the electrode 32 includes four electrodes 32A to 32D arranged in an array. Similarly to the elastic body 33, the elastic body 34 includes elastic bodies 34A to 34D that support the electrodes 32A to 32D, respectively. Each of the elastic bodies 34A to 34D includes four springs. The set of the electrode 32 and the elastic body 34 has a structure in which the set of the electrode 12 and the elastic body 14 (springs 14A to 14D) of the first embodiment is divided into small pieces, and four small sets are arranged in an array. Therefore, the detailed description of the electrodes 32A to 32D and the elastic bodies 34A to 34D is similar to that provided above.

In the present embodiment, the wirings L1 to L4 that electrically connect the electrodes 31A to 31D and the circuit board 16 (more specifically, wiring patterns P1 to P4 of the circuit board 16) are provided separately from the elastic body 33. In the present embodiment, the wirings L5 to L8 that electrically connect the electrodes 32A to 32D and the circuit board 16 (more specifically, wiring patterns P5 to P8 of the circuit board 16) are provided separately from the elastic body 34. With such a configuration, the potential difference between the electrodes 31 and 32 is input to the processing circuit 16A via the wirings L1 to L8. As in the first embodiment, the electrodes 31A to 31D and 32A to 32D may be electrically connected to the circuit board 16 by one or a plurality of springs that serve as corresponding ones of the elastic bodies 33A to 33D and 34A to 34D that support the electrodes.

In this embodiment, the processing circuit 16A may average the potentials of the electrodes 31A to 31D and average the potentials of the electrodes 32A to 32D, and a signal of a potential difference between the potential of the electrode 31 and the potential of the electrode 32 obtained by the averaging may be set as a target of processing of extracting a biological signal. With such a configuration, reduction of random noise can be expected. More specifically, among the noise components, the noise component having no correlation between the electrodes 31 and 32 is reduced by being divided by the square root of the number of electrodes (here, four) by the averaging, and the SN ratio is improved by the square root times.

In addition, it is also possible to regard the two-dimensionally arranged electrodes 31A to 31D as reception antennas, and four parts corresponding to (for example, facing) the electrodes 31A to 31D of the human body H as transmission antennas.

In such a case, the processing circuit 16A may derive the potentials of the four parts by multiple input multiple output (MIMO) in which the electrodes 31A to 31D are reception antennas and the four parts are transmission antennas. For example, the processing circuit 16A demodulates the potentials of the four parts based on the actual potentials of the electrodes 31A to 31D. As an example, the processing circuit 16A multiplies a column of the potentials of the electrodes 31A to 31D by an inverse matrix of a matrix of specific values obtained in advance by an experiment or the like to derive potentials of four parts. The processing circuit 16A may acquire the potentials of the four parts obtained in this manner as potentials representing the biological signals acquired by the electrodes 31A to 31D (potentials with reduced noise), and acquire the potential of the electrode 31 on the basis of these potentials.

As another example, the processing circuit 16A may acquire the potentials of the four parts as potentials (potentials with reduced noise) representing the biological signals acquired by the electrodes 31A to 31D by multivariate analysis such as blind source separation with the electrodes 31A to 31D as reception antennas and the four parts as transmission antennas, and acquire the potential of the electrode 31 based on these potentials.

The processing circuit 16A can also acquire the potential of the electrode 32 by a similar method.

As described above, the processing circuit 16A may acquire the potential of the electrode 31 by at least one of MIMO and multivariate analysis with the electrodes 31A to 31D as reception antennas and the four parts corresponding to the electrodes 31A to 31D of the human body H as transmission antennas. The processing circuit 16A further may acquire the potential of the electrode 32 by at least one of MIMO and multivariate analysis with the electrodes 32A to 32D as reception antennas and the four parts corresponding to the electrodes 32A to 32D of the human body H as transmission antennas. As a result, the noise component of the signal represented by the potential difference between the electrode 31 and the electrode 32 is reduced to improve the SN ratio of the signal. These methods are particularly suitable for a noise source that do not overlap uniformly at the electrodes 31 and 32, such as a motion artifact. Furthermore, as will be described later, even when the biological signal is a biological signal representing an electromyogram, a noise source that is not uniformly superimposed on the electrodes 31 and 32 may occur, and thus the above method is effective.

### Third Embodiment

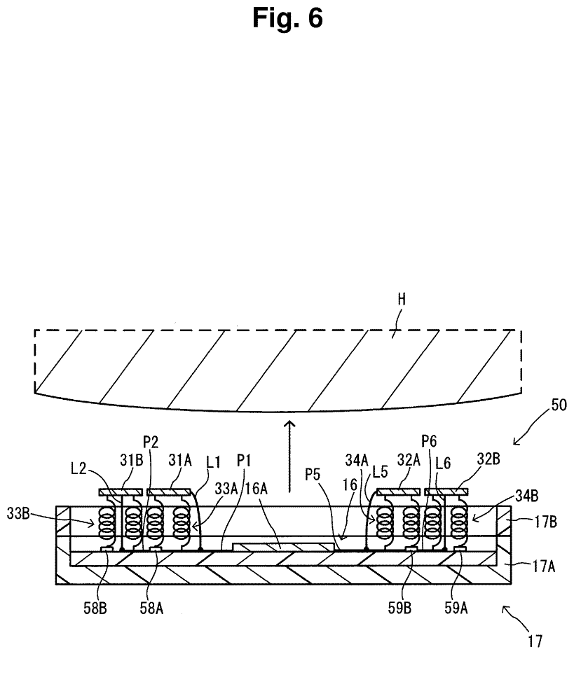

A biological signal acquisition device 50 according to a third embodiment is configured as illustrated in FIG. 6, for example, and detects each of contact areas of the electrodes 31A to 31D and the electrodes 32A to 32D with the human body H. Based on the detected contact areas, the biological signal acquisition device 50 acquires a biological signal using only an electrode having a contact area larger than a predetermined reference (an electrode in a good contact state) among the electrodes 31A to 31D and the electrodes 32A to 32D. Hereinafter, differences from the first embodiment and the second embodiment will be described. For the description not mentioned below, the description of the first embodiment and the second embodiment is referred to as appropriate.

The biological signal acquisition device 50 further includes pressure sensors 58A to 58D and 59A to 59D (only a part thereof is illustrated in FIG. 6) provided corresponding to the electrodes 31A to 31D and 32A to 32D, respectively, used to detect the contact areas. The pressure sensors 58A to 58D and 59A to 59D include piezoelectric elements or the like. The pressure sensor 58A is disposed between a spring included in the elastic body 33A supporting the corresponding electrode 31A and the circuit board 16, and is pressed by contraction of the spring when the electrode 31A comes into contact with the human body H. The pressure sensor 58A detects the pressure on the electrode 31A of the human body H by detecting the pressing force. Here, the contact area between the human body H and the electrode 31A increases as the pressure increases. Conversely, the lower the pressure, the smaller the contact area (including non-contact area). Therefore, here, the contact area is detected by detecting the pressure. The same applies to other pressure sensors. Each of the pressure sensors (for example, the pressure sensor 58A) may be provided at any position where the pressure due to the contact of the corresponding electrode (for example, the electrode 31A) with the human body H can be detected.

Each of the pressure sensors 58A to 58D and 59A to 59D is electrically connected to the circuit board 16. The circuit board 16 electrically connects, with the processing circuit 16A, an electrode corresponding to a pressure sensor that has detected a pressure exceeding a predetermined value among the pressure sensors 58A to 58D and 59A to 59D, that is, a contact area larger than a predetermined reference. The circuit board 16 does not electrically connect, with the processing circuit 16A, an electrode corresponding to a pressure sensor that has not detected a pressure exceeding a predetermined value, that is, that has detected a contact area equal to or less than a predetermined reference.

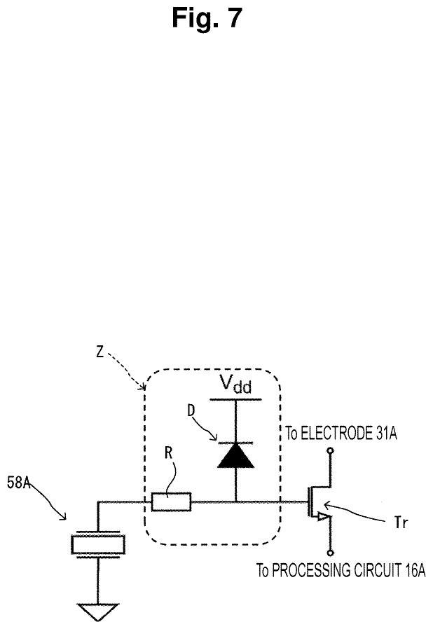

As such a circuit, an example of a circuit using the pressure sensor 58A is illustrated in FIG. 7. When the pressure sensor 58A detects a pressure due to contact of the human body H with the electrode 31A, the circuit of FIG. 7 outputs a voltage corresponding to the pressure. The output voltage is applied as a base voltage to the transistor Tr via a protection circuit Z. The transistor Tr is turned on when a base voltage indicating that the pressure exceeds a predetermined value is applied to electrically connect the electrode 31A and the processing circuit 16A. Note that the protection circuit includes a resistor R and a diode D, and protects the transistor Tr so that when an excessive voltage (a voltage exceeding Vdd) is generated from the pressure sensor 58A, the voltage is not applied to the base voltage. The circuit of FIG. 7 is provided for each of the pressure sensors 58B to 58D and 59A to 59D. With such a configuration, the contact area can be detected with less power consumption.

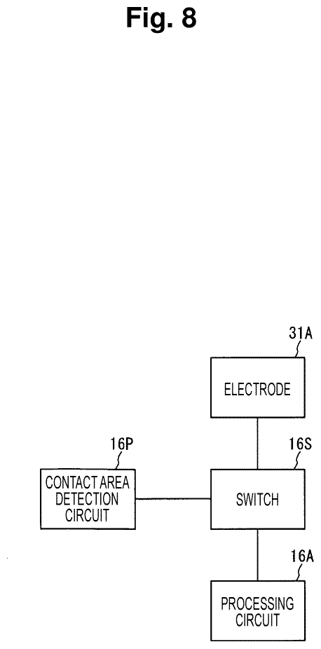

For example, a circuit including a contact area detection circuit 16P and a switch 16S illustrated in FIG. 8 may be mounted on the circuit board 16. The contact area detection circuit 16P detects a contact area between the electrode 31A and the human body H, and turns on the switch 16S in a case where the contact area is larger than a predetermined reference, thereby electrically connecting the electrode 31A and the processing circuit 16A. An example of the circuit of FIG. 8 is the circuit of FIG. 7. A circuit connecting the pressure sensor 58A and the transistor Tr in FIG. 7 corresponds to the contact area detection circuit 16P in FIG. 8, and the transistor Tr corresponds to the switch 16S. A similar circuit to that in FIG. 8 is provided for each of the electrodes 31B to 31D and 32A to 32D.

The contact area detection circuit 16P may be configured to detect a contact state between the electrode 31A or the like and the human body H using a conversion element that converts a physical quantity generated by contact between the electrode 31A or the like and the human body H in an analog manner into a voltage, a current, or the like, other than the pressure sensor. Note that, as for the conversion element (including the pressure sensor), bounce and chattering may become problems. Therefore, a conversion element having hysteresis such as a Schmitt trigger is used to suppress frequent switching of the switch so that the circuit operation of the circuit board 16 is more stable.

The contact area detection circuit 16P may detect the contact state by detecting the impedance between the body surface and the electrode for each of the electrodes 31A to 31D and 32A to 32D. A lower impedance indicates a larger the contact area. The contact area detection circuit 16P detects the impedance between the body surface and the electrode for each electrode by applying a voltage to or causing a current to flow through any plurality of electrodes of the electrodes 31A to 31D and 32A to 32D. In this method, the impedance itself can be used for determining whether the electrode is used for acquiring the biological signal, so that there is an advantage that detailed conditioning according to required measurement accuracy is possible. In addition, the contact area detection circuit 16P can also measure mental sweating as a method of measuring stress or tension, based on the detected impedance.

In a case where each of the electrodes 31 and 32 has a plurality of electrodes 31A to 31D or 32A to 32D, the contact area of a part of the electrodes may be reduced (including non-contact) due to the shape of the human body H to which the electrodes are applied. Such an electrode that has a small contact area, that is, that is not in contact with or is in contact with halfway with the human body H cannot measure a biological signal, and acquires only noise by serving as an antenna that picks up radio waves flying around the environment. According to the present embodiment, an electrode having a small contact area is not used to acquire a biological signal, so that the SN ratio of signals acquired by the electrodes 31 and 32 is improved.

The circuit board 16 may acquire the potentials of a plurality of electrodes in a good contact state by MIMO or multivariate analysis described in the second embodiment, thereby acquiring the biological signal. With this configuration, further improvement of the SN ratio can be expected.

### Modification

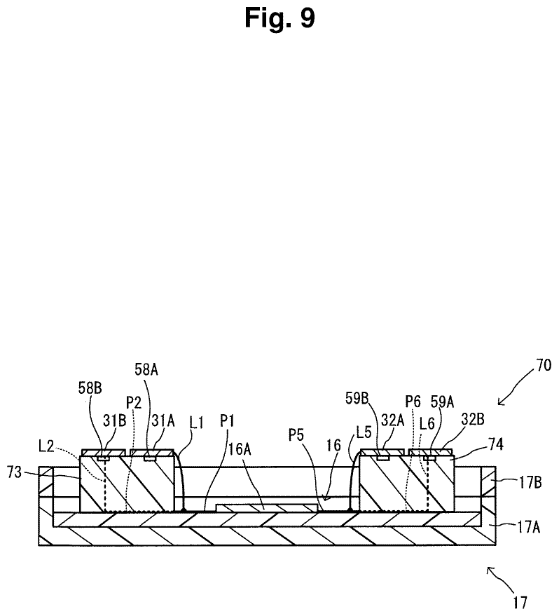

Various modifications are possible for each of the above embodiments. Any of the elastic bodies 13, 14, 33, and 34 may be made of sponge, natural rubber, synthetic rubber, or the like. For example, as in a biological signal acquisition device 70 illustrated in FIG. 9, instead of the elastic bodies 33 and 34, elastic bodies 73 and 74 each made of one elastic member such as sponge, natural rubber, or synthetic rubber that supports the plurality of electrodes 31A to 31D or 32A to 32D may be provided. As a result, the structure of the biological signal acquisition device 70 can be simplified as compared with the case where the springs are provided. The plurality of electrodes 31A to 31D of the electrode 31 preferably has flexibility, and the outer periphery of the entire electrodes 31A to 31D preferably fits within or coincides with the outer periphery of the elastic body 73 when viewed from the human body H side. With this configuration, the contact areas of the electrodes 31A to 31D are secured. The same applies to the electrode 32. The pressure sensor 58A and the like may be provided between the elastic body 73 and the electrode 31A or other positions. The pressure sensor 58A and the like may be omitted. The wiring L1 and the like may pass through the elastic body 73. The electrodes 11 and 12 may have, for example, a polygonal shape. The elastic body 13 may include a plurality of elastic bodies (springs or the like) that press the respective corners of the polygonal electrode 11 against the human body H. The elastic body 14 may include a plurality of elastic bodies (springs or the like) that press the respective corners of the polygonal electrode 12 against the human body H.

The specific configuration of the biological signal acquisition device is appropriately changed depending on what the biological signal is. Examples of the biological signal include an electromyographic signal, an electroencephalogram, and the like. The biological signal acquisition device may be adopted at a part to which the human body H naturally applies pressure, such as a device attached to the sole of a foot, a seat surface or a back surface of a chair or the like. With this configuration, the human body H can come into contact with the plurality of electrodes 31A to 31D or 32A to 32D while pressing the electrodes by its own weight, so that the contact areas with the electrodes increases, and the contact state with the electrodes is improved. The living body to be a subject of the biological signal acquisition device may be an animal other than a human.

The biological signal acquisition device only needs to include at least two electrodes, and may include three or more electrodes depending on the use. In this case, the configuration of the electrode 11 or the elastic body 13 may be adopted for each electrode. Note that the configuration described in the above embodiments (configuration in which an elastic body is provided, an electrode is configured by a plurality of electrodes, and the like) may be adopted for at least one of the at least two electrodes. By adopting the above configuration for one electrode, the effects described above can be obtained at least for the electrode. Any of the electrodes 11, 12, 31, and 32 may be connected to a reference potential.

The biological signal acquisition device only needs to include an electrode and an elastic body, and other members may be externally attached. In particular, an amplifier or the like that amplifies the biological signal may be provided in an external device electrically connected to the biological signal acquisition device. In this case, the elastic body may be fixed not to the circuit board but to a portion of, for example, a housing facing the electrode. Each set of the electrode and the elastic body may be provided in corresponding one of a plurality of separate housings that are attached to the living body or are in contact with the living body.

### SUPPLEMENTARY NOTES

Configurations disclosed in the present specification including those of the above embodiments and the like as examples will be stated below as supplementary notes.

### Supplementary Note 1

A biological signal acquisition device including: a first electrode and a second electrode configured to be in contact with a living body and configured to acquire a biological signal emitted by the living body when the first electrode and the second electrode are in contact with the living body; and a first elastic body configured to press the first electrode against the living body when the first electrode is in contact with the living body.

### Supplementary Note 2

The biological signal acquisition device according to Supplementary Note 1, further including a second elastic body that presses the second electrode against the living body when the second electrode is in contact with the living body.

### Supplementary Note 3

The biological signal acquisition device according to Supplementary Note 1 or 2, further including a circuit board that acquires a biological signal using the first electrode and the second electrode, in which the first elastic body electrically connects the first electrode and the circuit board.

### Supplementary Note 4

The biological signal acquisition device according to any one of Supplementary Notes 1 to 3, in which the first electrode has flexibility.

### Supplementary Note 5

The biological signal acquisition device according to any one of Supplementary Notes 1 to 4, in which the first electrode has a polygonal shape, and the first elastic body includes a plurality of elastic bodies that press respective corners of the first electrode against the living body when the first electrode is in contact with the living body.

### Supplementary Note 6

The biological signal acquisition device according to any one of Supplementary Notes 1 to 5, further including a circuit that acquires the biological signal using the first electrode and the second electrode, in which the first electrode includes a plurality of electrodes, and the circuit detects a contact area with the living body for each of the plurality of electrodes, and acquires the biological signal using only an electrode having the contact area larger than a predetermined reference among the plurality of electrodes.

### Supplementary Note 7

The biological signal acquisition device according to Supplementary Note 6, further including a pressure sensor used to detect the contact area.

### Supplementary Note 8

The biological signal acquisition device according to Supplementary Note 6 or 7, in which the circuit acquires the biological signal by multiple input multiple output (MIMO) using only a plurality of electrodes satisfying the predetermined reference, and using each of the plurality of electrodes as a reception antenna and a plurality of parts of the living body corresponding to the plurality of electrodes as transmission antennas, or by multivariate analysis.

### Scope

The present invention is not limited to the above-described embodiments and modifications. For example, the present invention includes various modifications to the above embodiments and modifications that can be understood by those skilled in the art within the scope of the technical idea of the present invention. The configurations described in the above embodiments and modifications can be appropriately combined inconsistency. It is also possible to delete any of the above-described components.

### REFERENCE SIGNS LIST

10, 30, 50, 70 Biological signal acquisition device
11, 12, 31, 31A to 31D, 32, 32A to 32D Electrode
13, 14, 33, 33A to 33D, 34, 34A to 34D, 73, 74 Elastic body
13A to 13D, 14A to 14D Spring
16 Circuit board
16A Processing circuit
16B, 16C Wiring pattern
16P Contact area detection circuit
16S Switch
58A to 58D, 59A to 59D Pressure sensor
H Human body
L1 to L8 Wiring
P1 to P8 Wiring pattern

1-8. (canceled)

9. A biological signal acquisition device comprising:
a first electrode and a second electrode configured to be in contact with a living body and configured to acquire a biological signal emitted by the living body when the first electrode and the second electrode are in contact with the living body; and
a first elastic body configured to press the first electrode against the living body when the first electrode is in contact with the living body.

10. The biological signal acquisition device according to claim 9, further comprising:
a second elastic body configured to press the second electrode against the living body when the second electrode is in contact with the living body.

11. The biological signal acquisition device according to claim 9, further comprising:
a circuit board configured to acquire a biological signal using the first electrode and the second electrode, wherein the first elastic body electrically connects the first electrode to the circuit board.

12. The biological signal acquisition device according to claim 9, wherein the first electrode has flexibility.

13. The biological signal acquisition device according to claim 12, wherein the first electrode has a polygonal shape, and the first elastic body includes a plurality of elastic bodies each configured to press respective corners of the first electrode against the living body when the first electrode is in contact with the living body.

14. The biological signal acquisition device according to claim 9, further comprising:
a circuit configured to acquire the biological signal using the first electrode and the second electrode, wherein the first electrode includes a first plurality of electrodes, and the circuit is configured to detect a contact area with the living body for each of the first plurality of electrodes.

15. The biological signal acquisition device according to claim 14, wherein the circuit is further configured to acquire the biological signal using only an electrode having a contact area larger than a predetermined reference among the first plurality of electrodes.

16. The biological signal acquisition device according to claim 14, further comprising:
a pressure sensor, wherein the circuit is configured to detect the contact area based on a measurement of the pressure sensor.

17. The biological signal acquisition device according to claim 14, wherein the circuit is configured to acquire the biological signal by multiple input multiple output (MIMO) using only a second plurality of electrodes of the first plurality of electrodes having a contact area larger than a predetermined reference, and using each of the second plurality of electrodes as a reception antenna and a plurality of parts of the living body corresponding to the second plurality of electrodes as transmission antennas, or by multivariate analysis.

18. A method comprising:
contacting a first electrode and a second electrode with a living body, wherein contacting the first electrode with the living body comprises pressing, by a first elastic body, the first electrode against the living body; and
acquiring, by the first electrode and the second electrode, a biological signal emitted by the living body when the first electrode and the second electrode are in contact with the living body.

19. The method according to claim 18, wherein contacting the second electrode with the living body comprises pressing, by a second elastic body, the second electrode against the living body.

20. The method according to claim 18, further comprising:
acquiring, by a circuit board, a biological signal using the first electrode and the second electrode, wherein the first elastic body electrically connects the first electrode to the circuit board.

21. The method according to claim 18, wherein the first electrode has flexibility.

22. The method according to claim 21, wherein the first electrode has a polygonal shape, and the first elastic body includes a plurality of elastic bodies each configured to press respective corners of the first electrode against the living body when the first electrode is in contact with the living body.

23. The method according to claim 18, wherein the first electrode includes a first plurality of electrodes, wherein the method further comprises:
detecting, by a circuit, a contact area with the living body for each of the first plurality of electrodes; and
acquiring, by the circuit, the biological signal using the first electrode and the second electrode, wherein the biological signal is acquired using only an electrode having a contact area larger than a predetermined reference among the first plurality of electrodes.

24. The method according to claim 23, wherein detecting the contact area comprises detecting the contact area based on a measurement of a pressure sensor.

25. The method according to claim 23, wherein the circuit is configured to acquire the biological signal by multiple input multiple output (MIMO) using only a second plurality of electrodes of the first plurality of electrodes having a contact area larger than a predetermined reference, and using each of the second plurality of electrodes as a reception antenna and a plurality of parts of the living body corresponding to the second plurality of electrodes as transmission antennas, or by multivariate analysis.
